(19) United States
(12) Patent Application Publication
(10) Pub. No.: US 2012/0265140 A1
(43) Pub. Date: Oct. 18, 2012

(54) Medical syrnge prime and cross-contamination free devices

(75) Inventors: Gale H. Thorne, JR., Bountiful, UT (US); Gale H. Thorne, Bountiful, UT (US)

(73) Assignee: Thorne Consulting and Intellectual Property, LLC, Bountiful, UT (US)

(21) Appl. No.: 13/066,565

(22) Filed: Apr. 18, 2011

(51) Int. Cl. A61M 5/315; A61M 5/36

(52) U.S. Cl. 604/125; 604/199

(57) ABSTRACT

An elastic shield for a conventional syringe is disclosed. In addition, two embodiments for providing a prime free syringe are disclosed. A first embodiment employs a tube which delivers liquid only from a liquid only zone within an associated syringe while a second embodiment is disclosed to discharge gas out of a proximal side of a plunger to void gas from a syringe delivery chamber.

### FIELD OF INVENTION

This invention generally relates to medical syringes and more specifically to safety devices which may be used with or added to conventional medical syringes to solve problems related to containing hazardous drug emissions or to reducing effects of cross contamination of materials which might be displaced from one side of a syringe plunger to the other side.

### BACKGROUND

It is well known by those skilled in the art of medical fluid infusion that use of conventional syringes can pose safety challenges when used to deliver hazardous drugs and when used to fill and deliver IV fluids to patients. When drawing a dose into a syringe from a vial containing a hazardous drug, it is well known that dangerous emissions of aerosols and liquids can occur upon spiking a vial. However, there are secondary effects not as well publicized which should also be considered. For example, while a plunger moved within the syringe to draw and expel a dose appears to wipe interior walls of the syringe barrel clean, trace amounts may be left on the barrel wall and thereafter exposed across the wiped face of the barrel. Similarly, for more conventional syringe use in drawing and delivering an IV dose to a patient, any contaminant on a syringe barrel over which a plunger is drawn in a filling process may be communicated in small amounts to liquid drawn into the syringe. For these reasons, cross contamination occurring due to back and forth displacement of a plunger within a syringe barrel is a concern. Such contamination may occur as a result of simply touching an associated barrel rod used to displace the plunger.

Further, each syringe, before delivery of a liquid, must be purged of gas (air) so that only liquids are delivered into a patient IV line. For this reason, it is common practice to prime air from each syringe prior to drug delivery into a patient IV line. This task, while performed so often that it has become transparent to clinicians, requires time and now that needleless connectors are commonly used, there may be increased danger of contamination due to liquid coming in contact with parts of a luer-lock fitting which surrounds the male insertion site of the fitting.

The cross contamination issue has been addressed by art which is exemplified by U.S. Pat. No. 7,175,609 issued Feb. 13, 2007 to Robin Scott Gray (Gray), U.S. Pat. No. 6,485,471 issued Nov. 26, 2002 to Maury Zivitz (Zivitz) and U.S. Patent Application US 2003/0097115 A! Filed Nov. 19, 2001 by Michael Gruenberg (Gruenberg). However, none of the cited art addresses a solution involving use of conventional syringes by which most drug transfer is performed.

The instant inventions disclosed herein address and provide solutions for both problems associated with hazardous drug delivery and cross contamination in conventional syringe use.

### Terms and Definitions

In the following table 1 is a list of terms and associated definitions provided to improve clarity and understanding of precepts of the instant invention:

TABLE 1
effluent, n: something that is emitted (flows out), particularly, from a vial.
fluid, n: a gas or liquid.
pre-filled syringe, n: a syringe, pre-filled with a predetermined volume
of solution.
HD, n: hazardous drug.
IV set, n: intravenous drug delivery tubing specifically dedicated for
use with an associated IV catheter and N container.
luer fitting, n: a medical connector which is in common use in medical
practice.
luer locking connector, n: a connector associated with a luer fitting having
a locking mechanism whereby a male and female connector are securely,
but generally releasibly affixed one to the other.
plunger, n: a piece which slides within a syringe barrel, generally designed
to keep fluids on each side of the plunger within the barrel disparate.
shield, n: a protective cover for a device or a portion thereof, esp. a part
which encloses a portion of a syringe to provide a barrier isolating the
device or portion from surrounding environment
unitized, adj: a plurality of separate parts permanently joined to be
used as a single unit.

### BRIEF SUMMARY AND OBJECTS OF THE INVENTION

In brief summary, novel inventions disclosed herein provide answers for known issues related to cross-contamination and priming of medical syringes. While problems associated with priming and cross contamination are separable, they are part of a combined set of syringe operations which must be carefully performed to assure safety in delivery of fluids to a patient.

While gas in a syringe is visually evident and traditionally purged for safety prior to dispensing liquid from the syringe to a patient IV line, material which is communicated across a plunger in a syringe as it is displaced to draw and dispense fluids from the syringe is usually communicated in trace amounts and is not so easily detected. However, it is well known in the medical art that cross-contamination along the inner wall of a syringe barrel does occur when a syringe plunger is displaced inside the syringe barrel.

For this reason, it is highly desirable to provide a barrel shield to maintain an enclosure for a syringe barrel so that no contaminating material is available for communication to the external environment from inside the closed chamber of a syringe as the plunger is displaced, or, in opposite manner, no contaminating material which may become resident on the inner barrel wall proximal to the plunger is available to be transmitted into the closed chamber across the plunger barrel wall interface. For these reasons, it is desirable to provide a barrel shield to provide safety from trace amounts of material resulting from a displaced plunger left on either side of the plunger. An instant invention, as disclosed herein, provides a sheath about the proximal barrel opening and displace-able plunger and rod assembly to guard against barrel contamination and exposure of trace material not wiped from the barrel wall as a plunger is displaced.

The shield, according to the instant invention disclosed herein, is a balloon-like device having a body made of elastic material. The body has a cross-section which is elongated toward a closed end and has a length which permits complete displacement of plunger and rod assembly along the syringe barrel wall. The body has a throat section which is sized and shaped to fit snugly about the exterior of the syringe barrel and yet be stretched about flanges which extend outward from the syringe barrel for digital access and a button which is generally affixed to the rod of the plunger and rod assembly. When the plunger is disposed well within the barrel, the shield is folded in accordion style between the button and flanges to permit ready access to flanges and button for displacing the plunger.

With the shield in place, the inner surface of the barrel, the rod and button are fully protected by the shield cover and tortuous path provided by the tightly affixed throat section distal from the flanges. Thus, the barrel inner surface and therefore the plunger remain contamination free after the shield is fully disposed upon the syringe. Of course, the inner portion of the shield should be sterilized as is the syringe, prior to being made ready for use.

Also, as disclosed herein, such a shield may be affixed to a syringe after sterilization of both components (field assembly of a conventional syringe and a protective shield). A method for affixing the shield to a conventional syringe involves providing access to the exterior of the shield without contaminating contact with either an inner sterile surface of the shield or critical sterile parts of the syringe during the act of engaging the shield. For facilely accomplishing attachment of the shield to the syringe while maintaining desired stability and sterility, a portion of the throat of the body is folded upon itself to provide a cuff into which a rigid tool may be inserted to stretch the body about syringe barrel, flanges and rod button.

Of course, the shield may be affixed to a syringe in production. In such a case, the shield may be used to protect barrel and plunger and rod assembly of a pre-filled syringe. As is well known in the pre-filled syringe art, such syringes are sometimes provided without external packaging to protect proximal parts of the syringe. If a plunger of such a syringe is displaced proximally (for example to test for patency of blood flow), the plunger may be drawn over a contaminated surface permitting cross-contamination without jeopardy.

Problems related to assuring no gas is transmitted to a patient line from a syringe are separately solved by two instant inventions as disclosed herein. In one inventive embodiment, gas resident in the closed syringe chamber is trapped therein and retained. Such is accomplished by an elongated tube affixed to and inwardly directed from the distal orifice of the syringe barrel to provide a closed flow path for liquid through the orifice from a liquid only zone within the barrel. As is known in the syringe art and disclosed in U.S. Pat. No. 7,789,862 (issued to Gale H. Thorne, Jr. et al., see FIGS. 14-25 (Thorne)), air under influence of gravity in a syringe which is mostly filled with liquid is constrained and disposed along sides and edges of the syringe wall and enclosing chamber ends. So disposed, air cannot exist within a central zone of the syringe, called a liquid only zone in Thorne. By disposing an exit orifice of the elongated tube in the liquid only zone, only liquid can be dispensed from the syringe. Note that since no gas can be dispensed from the syringe, the syringe becomes prime free.

In a second inventive embodiment, a special plunger assembly permits gas in the chamber adjacent the plunger to exhaust gas through the plunger. The special plunger assembly comprises a plunger part having a distally disposed hydrophobic material which provides an interface to fluid inside the chamber from which it is desired to exhaust gas. Proximal from the hydrophobic material part, the plunger further comprises a one-way valve which in combination with the hydrophobic part permits one flow of gas outwardly from the chamber when the plunger is acted upon by a force which increases pressure within the chamber to force gas through the hydrophobic part and one way valve. Note that no gas can enter into the chamber as all matter in the gaseous state is restricted to travel along outer surfaces of the inner wall of the syringe barrel and cylinder enclosing ends.

Accordingly, it is a primary object to provide a prime free pre-filled syringe.

It is also a primary object to provide a plunger for a syringe whereby gas may be extracted from the syringe chamber through a plunger proximal to the chamber.

It is another primary object to provide a barrel protector for a conventional syringe.

It is an object that the barrel protector be able to be assembled to the syringe in a field environment.

These and other objects and features of the present invention will be apparent from the detailed description taken with reference to accompanying drawings.

### BRIEF DESCRIPTION OF THE DRAWINGS

FIG. 1 is a PRIOR ART is a cross section of a syringe barrel and other parts including a rod and plunger assembly drawn toward a proximal end of the syringe barrel.

FIG. 2 is a frontal elevation of a balloon-like barrel shield.

FIG. 3 is a frontal elevation of the barrel shield seen in FIG. 2 with a segment of the mouth portion turned upon itself to form a cuff.

FIG. 4 is a frontal perspective of the mouth of the barrel shield seen in FIG. 2.

FIG. 5 is a perspective of barrel shield seen in FIG. 3.

FIG. 6 is a perspective of a tool used to affix the shield to a conventional syringe barrel

FIG. 7 is a perspective of a combination of the tool seen in FIG. 6 and shield seen in FIG. 5, with a portion of the tool inserted into the cuff of the shield.

FIG. 8 is frontal elevation of another PRIOR ART depiction of a conventional syringe wherein a rod and plunger assembly fully displaced into the barrel of the syringe.

FIG. 9 is a side elevation of the syringe seen in FIG. 8 and tool and shield seen in FIG. 7 with the shield in process being assembled about barrel and rod and plunger of the syringe with the tool.

FIG. 10 is a frontal elevation of a syringe with rod and plunger assembly fully displaced into the syringe barrel with a shield affixed thereto, the shield being folded in pleats.

FIG. 11 is a frontal elevation of the syringe and shield seen in FIG. 10 with the rod and plunger extended to also extend the shield.

FIG. 12 is a cross section of a conventional syringe barrel and other parts including a liquid only accessing part, also seen in cross section and a rod and plunger assembly of the associated syringe.

FIG. 13 is a schematic of a syringe assembly similar to that seen in FIG. 12, except the barrel of the syringe assembly has been modified to make a liquid only access part integral with the barrel of the syringe.

FIG. 14 is a schematic of another syringe assembly with only a portion of the liquid only access part made integral with the barrel of the syringe.

FIG. 15 is a schematic of the syringe assembly seen in FIG. 14 with a shield affixed to the syringe barrel.

FIG. 16 is a schematic of a conventional syringe barrel with a gas removing plunger affixed to a rod is disposed within the syringe barrel to trap fluid therein, a cap for the syringe being shown displaced from the syringe for clarity of presentation.

FIG. 17 is a cross section of the plunger seen in FIG. 16 magnified and shown as a separate part.

FIG. 18 is a schematic of the syringe seen in FIG. 16 with a shield affixed to the barrel of the syringe.

### DETAILED DESCRIPTION OF THE ILLUSTRATED EMBODIMENTS

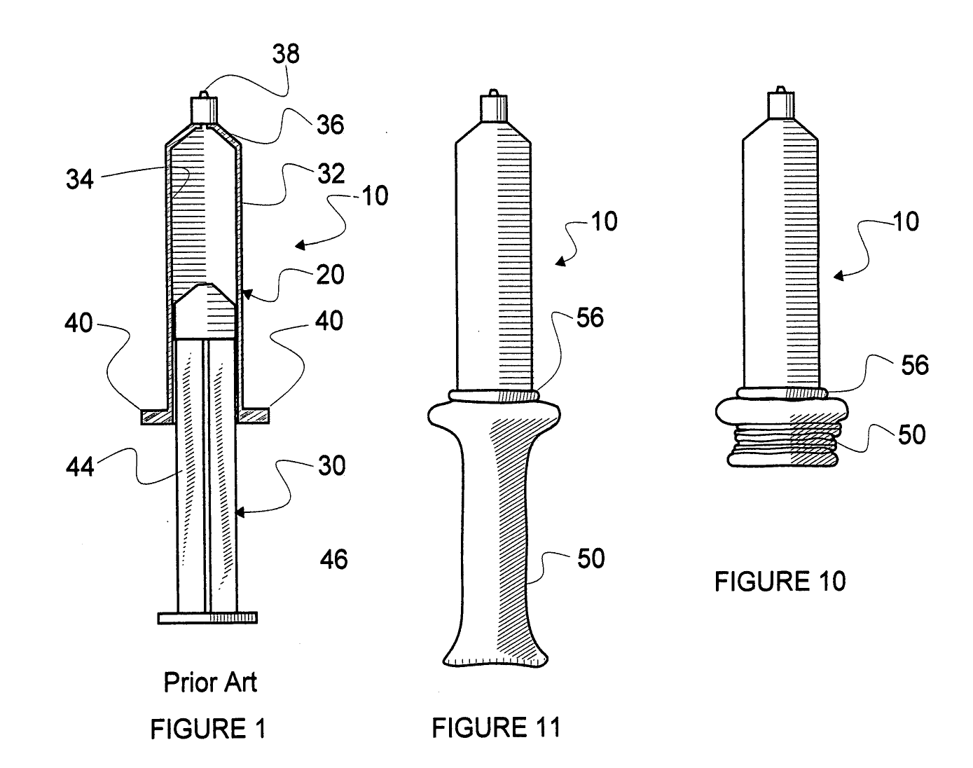

In this description, the term proximal is used to indicate the segment of the device normally closest to a user of the device. The term distal refers to the other end. Primes of numbers are used for parts which a similar, but not exactly the same as parts referenced by the base number. Reference is now made to the embodiments illustrated in FIGS. 1-18 wherein like numerals are used to designate like parts throughout.

For the most part, instant inventions disclosed herein are associated with and designed to augment function and performance of conventional syringes. An example of a conventional syringe 10 is seen as PRIOR ART in FIG. 1 to comprise a barrel 20 and a plunger and rod assembly 30. Further, barrel 20 comprises an elongated, concentric outer wall 32, which is inwardly opposed by an inner wall 34 diminished at an end 36 whereat is a fluid flow orifice 38. Outer wall 32 is further characterized by a pair of outwardly extending flanges, each numbered 40, usually used for digital control for operating syringe 10. Plunger and rod assembly 30 comprises a plunger 42 which is sized and shaped to wipe fluid from inner wall 34 as it is displaced to draw or dispense fluid associated with syringe 10 use. Also, plunger and rod assembly comprises an elongated rod 44 which is securely affixed to plunger 42 to facilitate displacement of plunger 42. Further, as is commonly true for conventional syringes, at a proximal end of rod 44 is a button 46 made available for grasping and displacing rod 44.

While it was stated supra that plunger 42 is sized and shaped to wipe fluid from inner wall 34, such wiping is not always complete and trace amounts of material have been found to be left as plunger 42 is moved back and forth in barrel 20. In such a case, any material which may be deleterious proximal to plunger 42 might be left for contamination of contents of the syringe between plunger 42 and orifice 38 as plunger 42 traverses the place of contamination when drawing fluid into syringe 10. Similarly, as plunger 42 traverses parts of inner wall 34, when dispensing fluid, trace amounts of the fluid may be left to contaminate portions of inner wall 34 proximal to plunger 42. Such contamination may represent significant danger in the case of Hazardous Drug handling and dispensing.

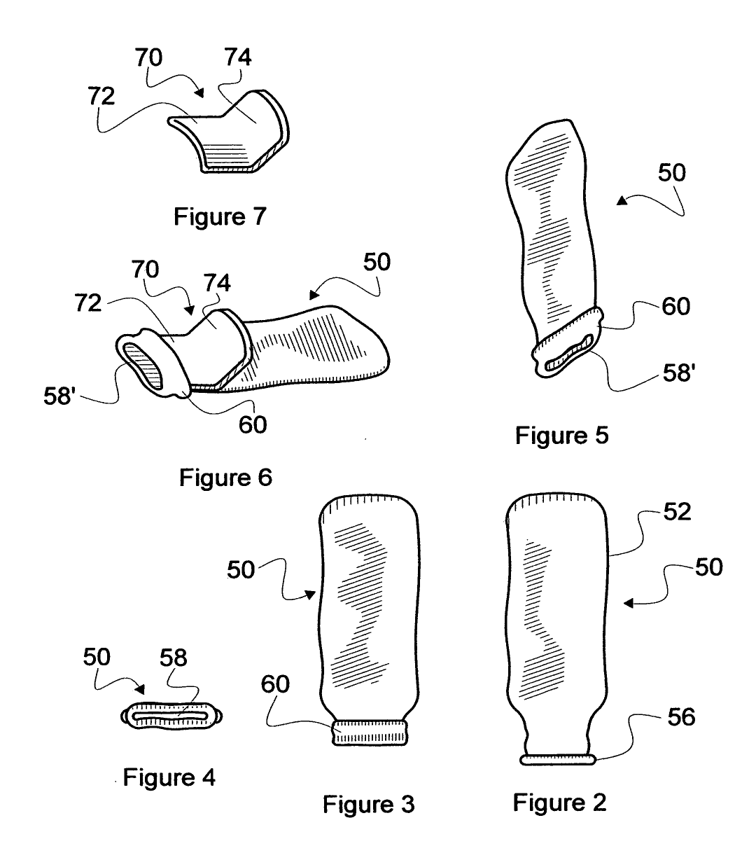

For these reasons, it is prudent to provide a shield for barrel 20 (and plunger and rod assembly 30). Such a shield 50 is seen in FIG. 2. Shield 50 is designed to provide a proximal cover for barrel 20 and plunger and rod assembly 30. While other such shields are known (see for example U.S. Pat. No. 7,175,609), none are known to the inventors to provide a shield for barrel and plunger and rod assembly of a conventional syringe and, further, none are known which can be affixed to provide a guard in a field of use environment.

Shield 50 comprises an elastic (preferably non-latex rubber), balloon-like sack having an elongated body 52 which is sized to slip over button 46. At an open or mouth end 54, shield 50 has a rolled section 56, similar to that of a balloon. As seen in FIG. 4, end 54 has an open mouth 58 having an inner wall circumference which is less than the circumference of an outer wall 32 of a syringe to which is to be affixed. Even so, the modulus of elasticity is permissive to providing a tight fit for mouth 58 about outer wall 32.

An important consideration is the need to retain a sterile condition inside shield 50 as such parts may contact elements of barrel 20 and plunger and rod assembly 30 which are and should remain uncontaminated. For this reason, shield 50 is provided sterilized with end 54 folded back upon itself, as seen in FIGS. 3 and 5, to provide a cuff 60. A new mouth 58′ is formed by cuff 60 To facilitate affixing shield 50 to syringe 10, a tool 70, as seen in FIG. 6, may be used. Tool 70 may comprise a cuff insertion portion 72 and a hand-held portion 74, which is substantially orthogonal to insertion portion 72.

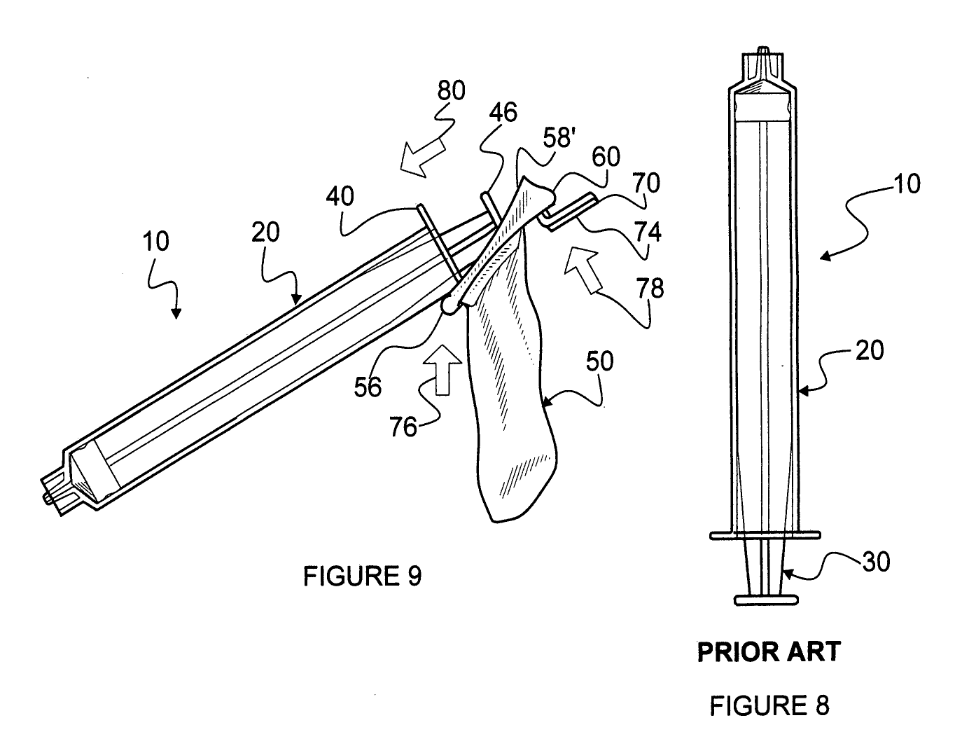

Reference is now made to FIGS. 7-11 wherein assembly and disposition of shield 50 on syringe 10 is seen. To affix shield 50 to syringe 10, plunger and rod assembly 30 is fully displaced into barrel 20 as seen in FIG. 8. Tool 70 is seen inserted into cuff 60 in FIG. 7.

One flange 40 of barrel 20 is inserted into mouth 58′ of shield 50 as seen in FIG. 9. Pressure is applied upon unfolded cuff in the region of rolled section 56 in a direction indicated by arrow 76. Using tool 70 cuff 60 is drawn about button 46 in direction of arrow 78 and further drawn downward about the other flange 40 in direction of arrow 80. Finally, with shield 50 in place, as seen in FIG. 11, rolled section 56 tightly embraces barrel 20 distal to flanges 40 (not seen). As seen in FIG. 10, shield 50 may be bunched or folded as an accordion when plunger and rod assembly 30 is fully displaced into barrel 20. Of course, shield 50 may be affixed more simply, especially if affixed before sterilization when contamination of the inner side of shield 50 is not an issue.

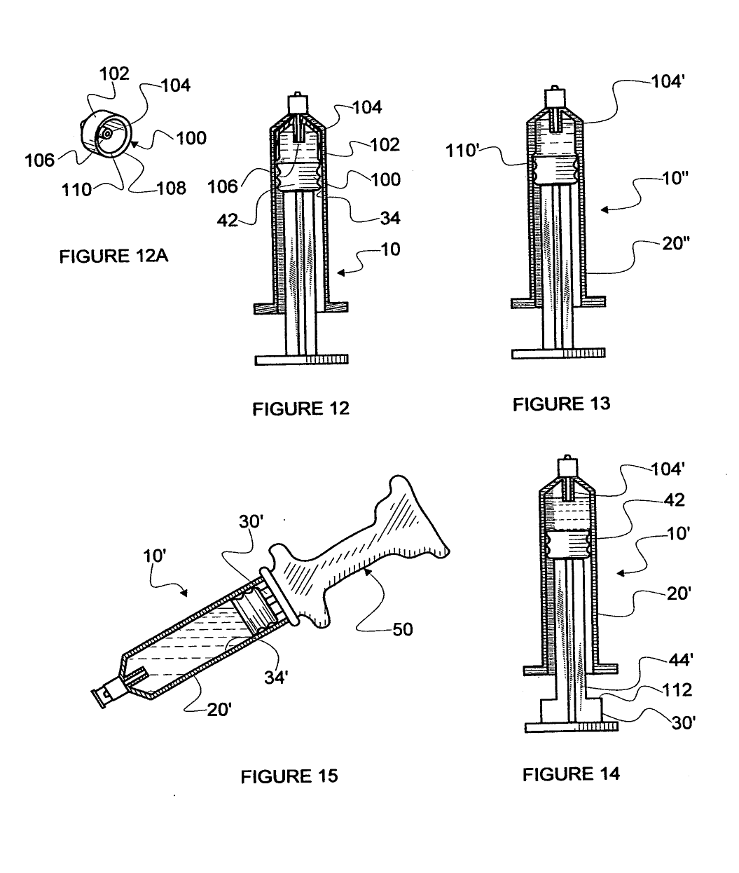

To satisfy the need for prime free operation of a syringe, a modification at orifice 38 may be made as seen by example in FIG. 12. To obviate a need to prime, a part 100 is inserted into barrel 20 as seen in FIG. 12. As may be seen in FIGS. 12 and 13, part 100 comprises a cylindrical wall 102 which is sized and shaped to conform with inner wall 34 of syringe 10. A medial elongated hollow tubular extension 104, having a thru hole 106 provides a communicating path from barrel 20 to orifice 38. Extension 104 opens the communicating path at a proximal site where only liquid can exist within syringe barrel 20 (a liquid only zone, reference is made to U.S. Pat. No. 7,789,862 for a full disclosure of the liquid only zone and acquisition of only liquid therefrom). To assure plunger does not proceed too far into barrel 10 to decrease total liquid volume and, thereby deliver gas into the liquid only zone, part 100 has a sidewall 108 which affords a stop 110 where beyond plunger 42 cannot be displaced into barrel 10.

Rather than inserting a special part, such as part 100, an elongated, hollow tube which communicates with orifice 38 may be molded into a barrel 20′ as seen in FIG. 14. In such a case, barrel 20′ of syringe 10′ is similar to barrel 20 except for elongated hollow tube 104′ which operates as tube 104 operates (see FIG. 12). Note, that a stop 112 is disposed on plunger rod 44′ of a plunger and rod assembly 30′ to limit travel of plunger 42 within barrel 20′ for the same purpose stop 110 is provided in part 100.

Another embodiment which is similar to embodiments seen in FIGS. 12 and 14 is seen in FIG. 13. However, in barrel 20″ in FIG. 13 a stop 110′ is molded therein.

Note, in FIG. 15 a shield 50 is disposed for protecting both barrel inner surface 34′ and plunger and rod assembly 30′. Similarly, shield 50 can be used to protect syringes seen in FIGS. 12 and 13.

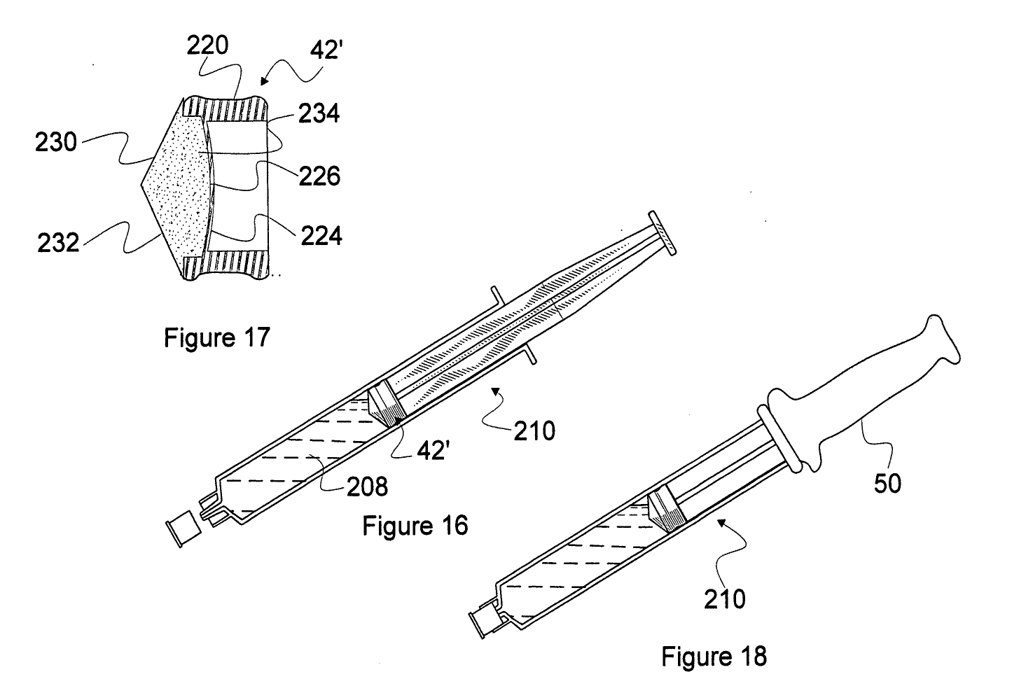

Instead of using a fluid pathway which only communicates with liquid from a liquid only zone as seen in FIGS. 12-14, gas can be separated from a delivery chamber 208 in a syringe 210 leaving only liquid using devices seen in FIGS. 16-18. Syringe 210 is similar to syringe 10 except for plunger 42′. Plunger 42′, seen magnified in FIG. 17, is made from two parts. A first part 220 is similar to a conventional plunger, having cylindrical inner barrel wall 34 interfacing material and geometry. However, rather than having a cone shaped portion which conforms to geometry of an orifice end of a syringe, a distally disposed portion of part 220 comprises a thin membrane 224 comprising a slit 226. A second part 230 is shaped on a distal side 232 to conform with associated syringe geometry and on the proximal side 234 to interface with membrane 224 to form a one way valve. In this manner, pressurized gas, imposed upon distal side 232, is proximally passed through slit 234, thus eliminating gas from chamber 208, leaving only liquid to be dispensed therefrom. It is important to note that gas emitted from chamber 208 may be contaminated by vapor of contents of chamber 208 and therefor should be contained. For this reason, a shield 50 is affixed to syringe 210 as seen in FIG. 18.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. A method for providing a shield against cross-contamination which occurs as insufficient material is wiped from a surface by a plunger of a conventional syringe displaced along an inner wall of a syringe barrel, said method comprising the steps of:
providing the conventional syringe comprising;
a circular barrel, having an extended length, an inner wall and an exterior wall and being of constant diameter and thickness, except for that variation required for a draw angle associated with molding the barrel;
said barrel further comprising a distally disposed opening through which fluid is drawn and dispensed and a proximal opening into which a plunger and plunger rod is inserted and displaced along the inner wall to facilitate fluid flow within the barrel;
a plunger and rod assembly sized to operate within the syringe barrel, said assembly comprising the plunger and a rod affixed to the plunger, said rod comprising a button affixed at the proximal end thereof for facilitating digital displacement of the plunger relative to the barrel;
said barrel still further comprising a pair of flanges which act as digital stops against which force is applied when displacing the plunger;
further providing a sheathe comprising:
a balloon-like device comprising;
a body made from elastic material, said body having a cross-section which is elongated toward a closed end, the elongated section being of sufficient length to permit displacement of an associated plunger rod to be fully displaced therein and said elongated section being unitized with a throat section which is smaller in cross-section than the elongated section and which has an insertable opening at the end opposite the elongated section;
said throat section being sized to snugly fit about the exterior wall of said barrel and said material having sufficient elasticity to permit the throat section to be stretched about the flanges and button to create a tortuous path about the exterior wall of the syringe and provide a protective sheath thereby;
displacing the device about the exterior wall, flanges and button of said syringe such that throat section is snugly disposed about the exterior wall distal to the flanges thereby forming the tortuous path for fluids and other matter disposed within the barrel; and
displacing the enlarged cross-section of the body over the button such that the enlarged section cooperates with displacement of the plunger rod to assure action of displacing the plunger rod does not either deleteriously affect barrel inner wall sterility or provide exposure of syringe contents outside the housing formed by syringe barrel and sheath.

2. The method according to claim 1 comprising the further steps of:
providing the syringe pre-sterilized;
providing the sheath pre-sterilized and having a portion of throat section folded upon itself to form a cuff;
providing a tool which can be inserted to said cuff to displace a portion of the sheath thereby;
inserting the tool into the cuff;
while holding a portion of the cuff opposite that portion of the cuff wherein the tool is inserted against the exterior wall of the barrel adjacent a first flange of the pair of flanges, using said tool to stretch and displace the sheath about the first flange, then the button and finally the second of the flanges such that the sheath is disposed to enclose the inner wall of the barrel and plunger and rod assembly without only pre-sterilized parts of the inner wall of the barrel and plunger and rod assembly being contacted by pre-sterilized parts of the sheath to enclose and ensure no external effects of cross-contamination.

3. The method according to claim 1 wherein the syringe providing step comprises providing a syringe pre-filled with a medical liquid.

4. Apparatus for removing gas from a chamber of a conventional syringe barrel, said apparatus comprising:
the syringe barrel comprising
a circular barrel having an inner wall of constant diameter and thickness, except for that variation required for a draw angle associated with molding the barrel;
said barrel further comprising a distally disposed opening through which fluid is drawn and dispensed and a proximal opening into which a plunger and plunger rod is inserted and displaced along the inner wall to facilitate fluid flow within the barrel;
said distal opening comprising a capable fitting;
a plunger and rod assembly sized and shaped to operate within the syringe barrel, said assembly comprising a plunger which is displaced to draw and dispense fluids through the distal opening;
said plunger comprising a section comprising cylindrical side wall which is sized and shaped to wipe said inner wall as the plunger is displaced within the barrel;
said plunger further comprising a portion which contacts fluid within the chamber, said portion comprising hydrophobic material which is impervious to liquid and porous to gas;
said section and portion comprising a common interface which forms a one way valve which permits flow of fluid only out of the chamber and which in combination with material of said portion only permits flow of gas from the chamber;
a cap sized and shaped to close the distally disposed opening such that sufficient pressure can be applied to fluid within the chamber to force gas through the hydrophobic material and one-way valve to remove gas from the chamber.

5. Apparatus according to claim 4 wherein said one-way valve comprises a slit disposed in a membrane disposed in the section.

6. A medical syringe comprising a barrel that is void of a valve assembly which is particularly characterized by a part which effects opening of the valve by being displaced proximally relative to the rest of the valve assembly, said part being so displaced upon contact with a distal end of the barrel to provide a fluid pathway from the syringe, said syringe further comprising:
the barrel comprising a distal orifice through which fluid is drawn and dispensed, a prime free barrel chamber and a proximal opening into which a plunger and plunger rod is inserted and displaced along the inner wall to facilitate fluid flow within the barrel;
a barrel closure which is provided to securely inhibit fluid flow from said barrel through said orifice, but which is opened to permit fluid communication from said barrel through said orifice;
fluid dispensed into the prime free chamber of said barrel, with said closure being disposed to inhibit flow, the plunger and plunger rod being displaced into said barrel to seal and contain said fluid therein;
said fluid comprising liquid for dispensing to a patient and a predetermined maximum amount of gas which is limited in volume by manufacturing process; and
an elongated tube disposed to provide a pathway for communicating liquid between said orifice and a liquid only zone within said prime free chamber of said barrel to thereby provide a path whereby only liquid can be dispensed from the prime free chamber through the orifice.

7. A syringe according to claim 6 wherein said syringe further comprises a sheath disposed about the barrel and plunger and plunger rod to protect sterility of items inside the barrel during displacement of the plunger and plunger rod relative to the barrel.
